(19) United States
(12) Patent Application Publication
(10) Pub. No.: US 2017/0268027 A1
(43) Pub. Date: Sep. 21, 2017

(54) METHOD FOR SYNTHESIS OF FATTY ACIDS

(71) Applicants: INSTITUT NATIONAL DES SCIENCES APPLIQUEES DE TOULOUSE, Toulouse Cedex 4 (FR); CENTRE NATIONAL DE LA RECHERCHE SCIENTIFIQUE, Paris Cedex 16 (FR); INSTITUT NATIONAL DE LA RECHERCHE AGRONOMIQUE, Paris Cedex 07 (FR); FIDOP (FONDS DE DEVELOPPEMENT DES FILIERES DES OLEAGINEUX ET DES PROTEAGINEUX), Paris (FR)

(72) Inventors: Julien CESCUT, Saint Michel de Lanes (FR); Jean-Louis URIBELARREA, Toulouse (FR); Stephane GUILLOUET, Vallegues (FR); Carole MOLINA-JOUVE, Toulouse (FR); Rana SAGNAK, Toulouse (FR)

(21) Appl. No.: 15/532,229

(22) PCT Filed: Dec. 21, 2015
(86) PCT No.: PCT/FR2015/053687

§ 371 Date: Jun. 1, 2017

(30) Foreign Application Priority Data
Dec. 22, 2014 (FR) 14/63149

(51) Int. Cl. C12P 7/64; C12N 15/82; A23L 33/12; C12N 15/81; A23K 20/158; A61K 31/20

(52) U.S. Cl. CPC C12P 7/6427; A23K 20/158; A61K 31/20; A23L 33/12; C12N 15/81; C12N 15/8247; A23D 9/00

(57) ABSTRACT

Disclosed is a method for synthesis of fatty acids by culturing a eukaryotic microorganism from the fungi kingdom, that is naturally oleaginous or rendered oleaginous. The culture is performed in the presence a fatty acid synthase inhibitor in the culture medium.

The present invention relates to a method for synthesis of fatty acids, especially short-chain or medium-chain fatty acids, from oleaginous eukaryotic microorganisms. The invention also relates to fatty acids as obtained by the method of the invention and use thereof in the fields of energy, chemistry, health, agri-food and nutrition.

The term “fatty acid” denotes molecules of carboxylic acid containing a hydrophobic carbon chain. The term “fatty acids” can denote the fatty acids stored in free form or in the form of triglycerides. They differ fundamentally in terms of the length of the carbon chain and the number of ethylenic bonds (degree of unsaturation), i.e. according to the potential existence of one or more double bonds between two adjacent carbon atoms. This leads to a differentiation between saturated fatty acids (which have no double bond) and mono- and polyunsaturated fatty acids (having one double bond and more than one double bond, respectively). The identification of the position of the ethylenic bond(s) makes it possible to differentiate the fatty acids having the same degree of unsaturation. Generally, the fatty acids have a carbon chain varying from 4 to 36 carbon atoms. Those having 2, 3 or 4 carbon atoms are referred to as volatile fatty acids, whereas those of which the carbon chain varies from 6 to 10 carbon atoms are referred to as short-chain fatty acids. The medium-chain fatty acids have a number of carbon atoms which varies between 12 and 14. Those of which the carbon chain varies from 16 to 18 carbon atoms are referred to as long-chain fatty acids, and those of which the carbon chain exceeds 18 carbon atoms are referred to as very long chain fatty acids. The most common have an even number of carbon atoms. The most common saturated fatty acids include hexadecanoic acid (C16:0), octadecanoic acid (C18:0), and eicosanoic acid (C20:0). Cis-9-tetradecanoic acid (C14:1(9)), cis-9-hexadecanoic acid (C16:1(9)) and cis-9-octadecanoic acid (C18:1(9)) belong to the group of monounsaturated fatty acids. Cis,cis-9-octadecadienoic acid (C18:2(9,12)) and cis,cis,cis,cis-5.8.11.14-eicosatetraenoic acid (C20:4(5,8,11,14)) are examples of polyunsaturated fatty acids. Fatty acids that are preferred in accordance with the invention are hexanoic acid (C6:0), heptanoic acid (C7:0), octanoic acid (C8:0), nonanoic acid (C9:0), decanoic acid (C10:0), dodecanoic acid (C12:0), and tetradecanoic acid (C14:0).

The term “eukaryotic microorganism from the fungi kingdom” denotes unicellular or multicellular organisms which have, in their cytoplasm, a plurality of organelles, and especially a ring, a Golgi apparatus, and mitochondria. These organisms reproduce either asexually by mitosis or sexually by meiosis when the culture conditions are unfavourable. By way of example, and without wishing to limit the scope of the present invention, the eukaryotic microorganisms from the fungi kingdom according to the present invention belong especially to the Yarrowia, Saccharomyces, Rhodotorula or Rhosporidium genus.

The “naturally oleaginous” eukaryotic microorganisms are considered to be the microorganisms from the fungi kingdom of which the metabolism enables the synthesis of fatty acids in quantities varying from 15% to 80% of dry microorganism (cell) mass. Fatty acids can be stored potentially in the form of triacylglycerides stored in lipid bodies.

The term “rendered oleaginous” denotes the eukaryotic microorganisms from the fungi kingdom of which the genome has been modified in order to increase the production of fatty acid (yield, rate, quantity, etc.) or to adjust the composition of accumulated fatty acids. This genetic modification can relate to at least one of the genes coding for the enzymes involved in the biosynthesis or storage of the fatty acids, especially the fatty acid synthase. The genetic modification can result from a gene suppression, insertion, deletion, substitution or duplication.

The term “fatty acid synthase inhibitor” denotes compounds capable of interacting with the active site of the fatty acid synthase or capable of interacting with another site of the fatty acid synthase before and/or after the fixation of said substrate to said enzyme at the active site thereof, wherein all of these interactions can be reversible or irreversible. This fixation results in a modulation of all or part of the enzyme activity of the fatty acid synthase. Among the inhibitors that are well known to a person skilled in the art, triclosan (5-chloro-2-(2,4-dichlorophenoxy) phenol), TOFA (5-(tetradecyloxy)-2-20 furancarboxylic acid), C75 (tetrahydro-4-methylene-2R-octyl-5-oxo-3S-furancarboxylic acid) and cerulenin (2,3-epoxy-4-oxo-7,10-hexadecadienoylamide) are used as fatty acid synthase inhibitors. Cerulenin is fixed irreversibly to the β-ketoacyl-ACP synthase and consequently inhibits the fatty acid synthase (Funabashi et al., 1989 Journal of Biochemistry 105, 751-55).

In yeasts and higher eukaryotes, the fatty acid synthesis reactions are performed by a protein homodimer which possesses all of the enzyme activities required to produce fatty acids and which is referred to as fatty acid synthase I (FAS I).

There exist eukaryotic microorganisms able to accumulate more than 30% of their dry mass in intracellular lipids in the presence of a carbon substrate. This is especially the case with Yarrowia lipolytica. Application FR 2 940 315 A1 discloses a culture method based on a controlled management of the carbon and nitrogen contents in the culture medium and which enables the synthesis of 0.35 glipid·g⁻¹ yeast dry mass; the synthesised fatty acids are fatty acids having, for the greater part, carbon chain lengths between C16:0 and C24:0. In another method for cultivating Yarrowia lipolytica (FR 2 981 363 A1), the controlled content of phosphorus, even in the presence of an excess of carbon substrate, allows the accumulation of polysaccharides and lipids at a rate of 50% in carbon of the biomass dry mass. Such methods, however, do not make it possible to modulate the profile of the fatty acids according to the envisaged use, especially according to the especial features of the field of application.

A certain number of specific inhibitors of the biosynthesis pathway of fatty acids are well known to a person skilled in the art. These are especially triclosan (5-chloro-2-(2,4-dichlorophenoxy) phenol), TOFA (5-(tetradecyloxy)-2-20 furancarboxylic acid), C75 (tetrahydro-4-methylene-2R-octyl-5-oxo-3S-furancarboxylic acid) and cerulenin (2,3-epoxy-4-oxo-7,10-hexadecadienoylamide).

Cerulenin (2,3-epoxy-4-oxo-7,10-hexadecadienoylamide) is a mycotoxin developed originally as an antifungal antibiotic and exerts an inhibitory effect on the activity of fatty acid synthase (FAS) [Nomura et al., 1972, J Antibiot (Tokyo) 25: 365-368]. Cerulenin is covalently bonded to a cysteine residue in the active site of β-ketoacyl-synthase, a condensation enzyme required for the synthesis of fatty acids [Price et al., 2001, J Biol Chem 276: 6551-6559].

Numerous studies have made it possible to analyse the effects of cerulenin on the growth of the microorganisms. Especially, Tanaka et al. have shown that cerulenin completely blocks the growth of a wild-type strain of Candida lypolytica when this grows on a substrate containing n-undecane or n-dodecane, but remains without exerting any effect when the substrate contains alkanes of larger size (n-tetradecane to n-octadecane) [Tanaka et al., European J. Appl. Microbiol., 1976, 3(2), 115-124]. More recently, Torella et al. have shown that the addition of cerulenin in the culture medium makes it possible to increase the yield of synthesis of specific short-chain fatty acids among the Escherichia coli strains. However, these results were obtained on strains genetically modified for at least one of the enzymes of the pathway for the biosynthesis of fatty acids [Torella et al., 2013, PNAS, 110(28), 11290-11295].

It is known that free short-chain fatty acids are toxic for microorganisms [Neal et al., 1965, J Bacteriol, 90(1), 126-131] and are therefore produced in small quantities by the wild-type strains in accordance with the natural pathways present in the metabolism.

When researching the modulation of the potential for biosynthesis of fatty acids induced by a nutritional limitation and with the addition of non-lethal doses of cerulenin, the inventors found, surprisingly, that the addition of cerulenin in the culture medium of a eukaryotic microorganism from the fungi kingdom, that is naturally oleaginous or rendered oleaginous, causes an increase in the accumulation of short-chain or medium-chain fatty acids in the yeast.

One aim of the present invention is to propose a method for producing short-chain or medium-chain fatty acids.

A further aim of the present invention is to provide short-chain or medium-chain fatty acids in large quantity and with an increased degree of purity.

An additional aim of the present invention is to provide short-chain or medium-chain fatty acids that can be used for biofuel uses.

Lastly, a further aim of the present invention is to provide short-chain or medium-chain fatty acids that can be used in the field of oleochemistry and/or the production of bioenergetics molecules and/or the preparation of a cosmetic and/or pharmaceutical and/or nutritional agent.

Oleochemistry relates to physico-chemical modifications applied to animal and vegetable oils and fats. It is thus present in a variety of fields of application, such as chemistry, materials, heath, and energy (lubricants, plastics, polymers, additives, biodiesels, etc.).

The present invention relates to a method for synthesis of short-chain or medium-chain fatty acids, preferably having between 4 and 15 carbon atoms, by culturing a eukaryotic microorganism from the kingdom of fungi, that is naturally oleaginous or rendered oleaginous, characterised in that the culture is performed in the presence of a fatty acid synthase inhibitor in the culture medium.

The addition of the aforementioned fatty acid synthase inhibitor in the culture medium causes a modulation of the elongation kinetics of fatty acids. The term “elongation kinetics of fatty acids” denotes the different reaction steps of the de novo synthesis cycle of fatty acids performed by FAS, especially transacetylation, transmalonysation, the step of condensation, and the two steps of reduction following the step of dehydration. The process of elongating the chain of fatty acyl is performed by successive cycles using the same steps of condensation of malonyl-CoA and acyl-ACP followed by steps of decarboxylation, reduction, and dehydration. The FAS releases palmityl-coA (16 carbon atoms) in the cytoplasm. In order to synthesise fatty acids having more than 16 carbon atoms, cytoplasm enzymes separate from FAS catalyse the same reactions as FAS in succession.

The present invention relates especially to a method for synthesis of short-chain or medium-chain fatty acids by culturing a eukaryotic microorganism from the kingdom of fungi, that is naturally oleaginous or originates from the yeast strain JMY3501, characterised in that the culture is performed in the presence of a fatty acid synthase inhibitor in the culture medium.

The strain of Yarrowia lipolytica JMY3501 is a strain genetically modified so as to optimise the accumulation of lipids, the culture conditions and the conditions for obtaining said strain being described in (Lazar Z et al., Metabolic Engineering 26 (2014) 89-99).

The present invention relates especially to a method for synthesis of short-chain or medium-chain fatty acids by culturing a eukaryotic microorganism from the kingdom of fungi, that is naturally oleaginous, characterised in that the culture is performed in the presence of a fatty acid synthase inhibitor in the culture medium.

The present invention relates more especially to a method as described above, characterised in that the short or medium chain of the fatty acids has between 4 and 15 carbon atoms.

The present invention also relates to a method for synthesis of short-chain or medium-chain fatty acids, preferably having between 4 and 15 carbon atoms, by culturing a eukaryotic microorganism from the kingdom of fungi, that is naturally oleaginous or rendered oleaginous, in which method the fatty acid synthase inhibitor is preferably selected from cerulenin and analogues thereof, triclosan (5-chloro-2-(2,4-dichlorophenoxy) phenol), TOFA (5-(tetradecyloxy)-2-20 furancarboxylic acid), bischloroanthrabenzoxocinone, thiolactomycin, platensimycin and also the analogues of these molecules, preferably C75 (4-methylene-2-octyl-5-oxo-tetrahydrofuran-3-carboxylic acid), C93 (or FAS93), and FAS31.

It should be noted that the compound C75 can be referenced in the literature especially under the following formulas: 4-methylene-2-octyl-5-oxo-tetrahydrofuran-3-carboxylic acid, tetrahydro-4-methylene-2R-octyl-5-oxo-3S-furancarboxylic acid or trans-4-carboxy-5-octyl-3-methylene butyrolactone.

In the present method, it is also possible to use any compound capable of inhibiting fatty acid synthase, such as orlisatat (N-formyl-L-leucine (1S)-1-[[(2S,3S)-3-hexyl-4-oxo-2-oxetanyl]methyl]dodecyl ester), GSK837149A (dibenzenesulfonamide urea), isoniazid, platencin, pyrazinamide, ethionamide, diazoborine, hexachlorophene, diclofenac, epigallocatechin-3-gallate (EGCG), luteolin, taxifolin, kaempferol, quercetin, apigenin, anthecotulide, anthecularin, 4-hydroxyanthecotulide and 4-acetoxyanthecotulide.

It should be noted that the orlistat compound can be referenced in the literature especially under the following formulas: N-formyl-L-leucine (1S)-1-[[(2S,3S)-3-hexyl-4-oxo-2-oxetanyl]methyl]dodecyl ester and (S)—((S)-1-((2S,3S)-3-hexyl-4-oxooxetan-2-yl)tridecan-2-yl)2-formamido-4-methylpentanoate.

Further fatty acid synthase inhibitors that can be used are those listed in the international application WO 2013/022 927, especially C247 and the molecules carrying a 3-aryl-4-hydroxyquinolin-2(1H)-one function, as described in the application WO 2007/089 634 filed by Merck, or those carrying a bisamide function, such as those described in the application WO 2008/059 214 filed by AstraZeneca.

The present invention also relates to a method for synthesis of short-chain or medium-chain fatty acids, preferably having between 4 and 15 carbon atoms, by culturing a eukaryotic microorganism from the kingdom of fungi, that is naturally oleaginous or rendered oleaginous, in which method the fatty acid synthase inhibitor is preferably selected from cerulenin and analogues thereof, triclosan (5-chloro-2-(2,4-dichlorophenoxy) phenol), TOFA (5-(tetradecyloxy)-2-20 furancarboxylic acid), bischloroanthrabenzoxocinone, thiolactomycin, platensimycin and also the analogues of these molecules, preferably C75 (4-methylene-2-octyl-5-oxo-tetrahydrofuran-3-carboxylic acid), C93 (or FAS93), FAS31, orlistat (N-formyl-L-leucine (1S)-1-[[(2S,3S)-3-hexyl-4-oxo-2-oxetanyl]methyl]dodecyl ester), GSK837149A (dibenzenesulfonamide urea), isoniazid, platencin, pyrazinamide, ethionamide, diazoborine, hexachlorophene, diclofenac, epigallocatechin-3-gallate (EGCG), luteolin, taxifolin, kaempferol, quercetin, apigenin, anthecotulide, anthecularin, 4-hydroxyanthecotulide, 4-acetoxyanthecotulide, and C247, wherein the fatty acid synthase inhibitor is more preferably cerulenin.

The present invention also relates to a method as described above, characterised in that the fatty acid synthase inhibitor is selected from cerulenin and analogues thereof, triclosan (5-chloro-2-(2,4-dichlorophenoxy) phenol), TOFA (5-(tetradecyloxy)-2-20 furancarboxylic acid), bischloroanthrabenzoxocinone, thiolactomycin, platensimycin and also the analogues of these molecules, preferably C75 (4-methylene-2-octyl-5-oxo-tetrahydrofuran-3-carboxylic acid), C93 (or FAS93), FAS31, orlistat (N-formyl-L-leucine (1S)-1-[[(2S,3S)-3-hexyl-4-oxo-2-oxetanyl]methyl]dodecyl ester), GSK837149A (dibenzenesulfonamide urea), isoniazid, platencin, pyrazinamide, ethionamide, diazoborine, hexachlorophene, diclofenac, epigallocatechin-3-gallate (EGCG), luteolin, taxifolin, kaempferol, quercetin, apigenin, anthecotulide, anthecularin, 4-hydroxyanthecotulide, 4-acetoxyanthecotulide, and C247.

The present invention also relates to a method as described above, characterised in that the fatty acid synthase inhibitor is cerulenin.

The present invention also relates to a method for synthesis of short-chain or medium-chain fatty acids, preferably having between 4 and 15 carbon atoms, by culturing a eukaryotic microorganism from the kingdom of fungi, that is naturally oleaginous or rendered oleaginous, in which method said microorganism is of the Yarrowia, Saccharomyces, Rhodotorula, or Rhodosporidium genus.

In an especial embodiment of the method according to the invention, said microorganism is the yeast Yarrowia lipolytica or Rhodotorula glutinis.

In an especial embodiment of the method according to the invention, said microorganism is the yeast Yarrowia lipolytica.

In another especial embodiment of the method according to the invention, said fatty acid synthase inhibitor is cerulenin.

In another especial embodiment of the method according to the invention, the cerulenin is introduced to the culture medium by pulsed addition, one-time addition, or multiple and successive additions.

Pulsed addition corresponds to an addition to the culture medium of a precise amount of cerulenin. This precise amount is proportional to the amount of microorganism present in the culture medium. The addition is performed within a very short space of time (a few seconds), which corresponds to the definition of the pulse. The effect of the cerulenin may disappear over time, and just one addition or a number of additions can be made. The time between two pulsed additions is dependent on the dynamics of the reduction of the effect of the cerulenin.

In another more especial embodiment of the method according to the invention, the cerulenin is introduced by continuous addition to the culture medium.

This flow rate is dependent on the concentration of microorganisms present, this concentration evolving continuously and the range of the flow rate thus being broad:

from 0.001 g_{ceru}·h⁻¹ to 20 g_{ceru}·h⁻¹, especially from 0.001 g_{ceru}·h⁻¹ to 10 g_{ceru}·h⁻¹, more especially from 0.4 mg_{ceru}·h⁻¹ to 20 mg_{ceru}·h⁻¹, even more especially from 0.4 g_{ceru}·h⁻¹ to 10 g_{ceru}·h⁻¹.

In another even more especial embodiment of the method according to the invention, the concentration of cerulenin varies from 0.01 to 25 mg/g of dry yeast, preferably from 0.01 to 14 mg/g of dry yeast, and more preferably from 0.05 to 14 mg/g of dry yeast.

In another even more especial embodiment of the method according to the invention, the concentration of cerulenin varies from 1 to 25 mg/g of dry yeast, and preferably from 1 to 14 mg/g of dry yeast.

In another even more especial embodiment of the method according to the invention, the concentration of cerulenin varies from 0.01 to 1 mg/g of dry yeast, preferably from 0.05 to 1 mg/g of dry yeast.

In the method according to the invention it is also conceivable to control other parameters, such as the content of FAS inhibitor.

Especially, it could be beneficial to keep the ratio between the rate of carbon consumption and the rate of nitrogen consumption (rC/rN) at a value between 5 and 100 moles of carbon consumed per mole of nitrogen consumed and preferably between 12 and 100 moles of carbon consumed per mole of nitrogen consumed.

Especially, it could be beneficial to keep the ratio between the rate of carbon consumption and the rate of nitrogen consumption (rC/rN) at a value between 16 and 100 moles of carbon consumed per mole of nitrogen consumed, preferably a value between 16 and 50 moles of carbon consumed per mole of nitrogen consumed.

Especially, it could be beneficial to keep the ratio between the rate of carbon consumption and the rate of nitrogen consumption (rC/rN) at a value between 12 and 50 moles of carbon consumed per mole of nitrogen consumed, preferably from 12 to 16 moles of carbon consumed per mole of nitrogen consumed.

Especially, it could be beneficial to keep the ratio between the rate of carbon consumption and the rate of nitrogen consumption (rC/rN) at a value between 12 and 16 (moles of carbon consumed per mole of nitrogen consumed).

In addition, in the method according to the invention, the content of phosphorus in the culture medium could be adjusted so as to keep the level of intracellular phosphorus of the yeast at a value varying from 4 to 27 mg/g of biomass.

In addition, the method according to the invention is characterised in that the short-chain or medium-chain fatty acids, preferably having between 4 and 15 carbon atoms, are obtained in the form of a mixture of free fatty acids and triglycerides.

The invention also relates to short-chain or medium-chain fatty acids that can be obtained by the method described above, such as pentanoic acid (C5:0), hexanoic acid (C6:0), heptanoic acid (C7:0), octanoic acid (C8:0), nonanoic acid (C9:0), decanoic acid (C10:0), dodecanoic acid (C12:0), tetradecanoic acid (C14:0), and pentadecanoic acid (C15:0).

The use of short-chain or medium-chain fatty acids, preferably having between 4 and 15 carbon atoms, obtained by the method of the invention can be provided in many separate technical fields.

The short-chain or medium-chain fatty acids, preferably having between 4 and 15 carbon atoms and as obtained by the method according to the invention, could be used in the field of oleochemistry, for example for the production of lubricants, surfactants, solvents, plasticisers, or polymers for adhesive, paint, glue, packaging, foam or coating applications.

Especially, the short-chain or medium-chain fatty acids, preferably having between 4 and 15 carbon atoms and as obtained by the method according to the invention, could be used for the production of energy molecules, especially for the production of biofuels.

The fuels for aviation have a carbon chain length focussed on C12 and C14; it is thus preferable to obtain oils having carbon chain lengths between C8 and C16, preferably close to C12 and C14 so as to reduce the energy cost of the post-treatment of the oils making it possible to obtain the alkanes.

More especially, the short-chain or medium-chain fatty acids, preferably having between 4 and 15 carbon atoms and as obtained by the method according to the invention, can be used for the cosmetic treatment of the skin or hair.

The fatty acids as obtained by the method of the invention can be used in the composition of shampoos, creams, gels and masks.

Even more especially, the short-chain or medium-chain fatty acids, preferably having between 4 and 15 carbon atoms and as obtained by the method according to the invention, can be used in the field of health and nutrition, for example for use in the form of pharmaceutical drugs.

Medium-chain triglycerides (MCTs) are recommended in some cases in order to rebalance the diet of individuals suffering from problems regarding the absorption of fats. These MCTs facilitate the co-absorption of liposoluble nutrients, such as vitamins A, D, E and K, or carotenoids.

The drawings and the following examples aim to further illustrate the present invention, without limiting the scope of the invention in any way.

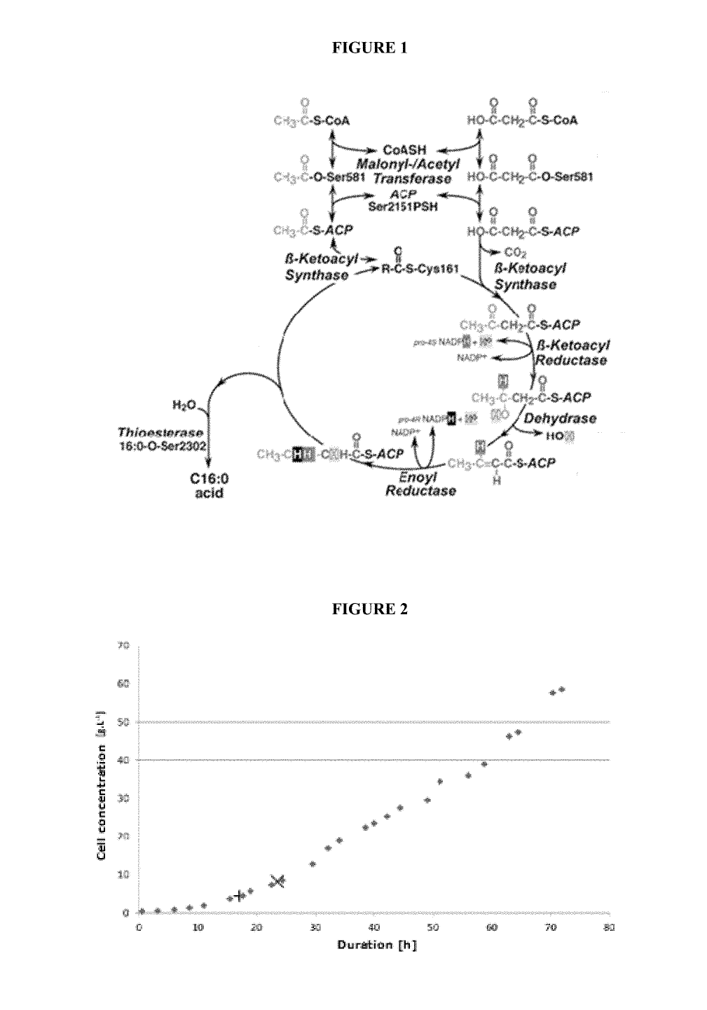

FIG. 1: Detail of the central anabolism of the fatty acids in the case of Yarrowia lipolytica.

FIG. 2: Development of the concentration of biomass (gₓ·l⁻¹) over time (hours, h) during the culture in fed-batch mode of Y. lipolytica with introduction of a nitrogen limitation (symbol +) and injection of a pulse of DMSO (10 mL) (symbol X) (Culture A).

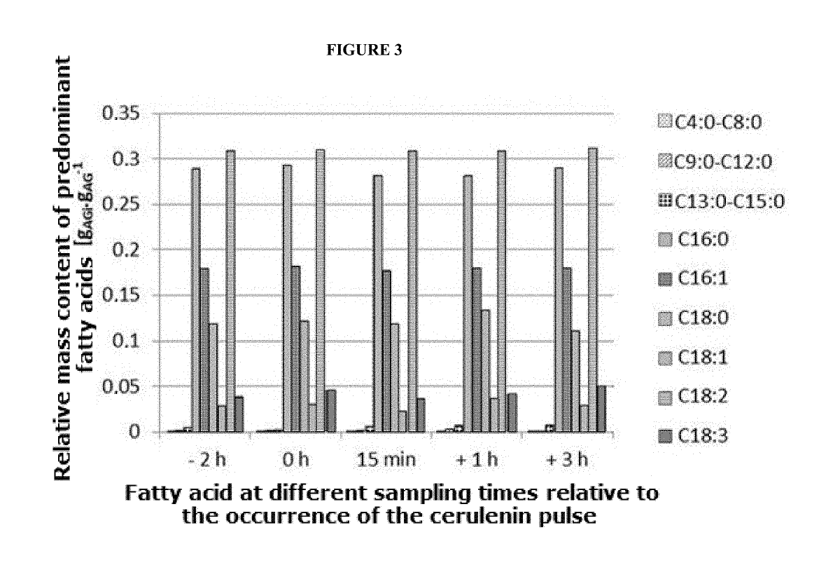

FIG. 3: Development of the fatty acids profile during the phase of lipid accumulation before (−2 h), during (0 h) and after (15 mn, 1 h, 3 h) a pulse of DMSO (10 mL) during a fed-batch culture of Y. lipolytica (Culture A).

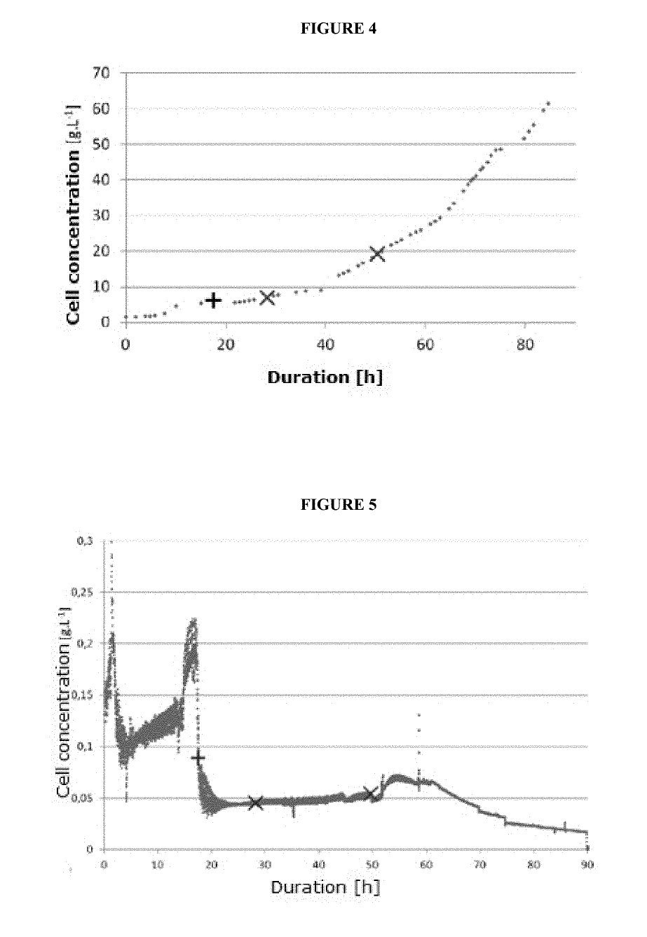

FIG. 4: Development of the concentration of biomass (gₓ·l⁻¹) over time (h) during the culture in fed-batch mode of Y. lipolytica with the introduction of a nitrogen limitation (symbol +) and the injection of pulses of cerulenin of 7 mg_{cerulenin}·gₓ⁻¹ (symbol X) (Culture B).

FIG. 5: Development of the rate of growth calculated on the basis of data of the capacitance probe [h−1] over time [h] during the culture in fed-batch mode of Y. lipolytica with the introduction of a nitrogen limitation (symbol +) and the injection of pulses of cerulenin of 7 mg_{cerulenin}·gₓ⁻¹ (symbol X). (Culture B)

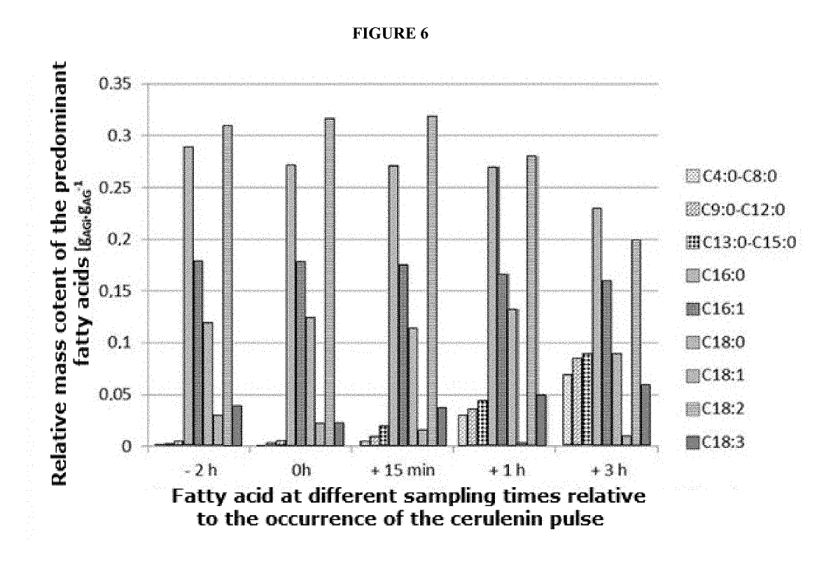

FIG. 6: Development of the fatty acids profile during the phase of lipid accumulation before (−2 h), during (0 h) and after (15 mn, 1 h, 3 h) a pulse of 7 mg_{cerulenin}·gₓ⁻¹ during a fed-batch culture of Y. lipolytica. (Culture B)

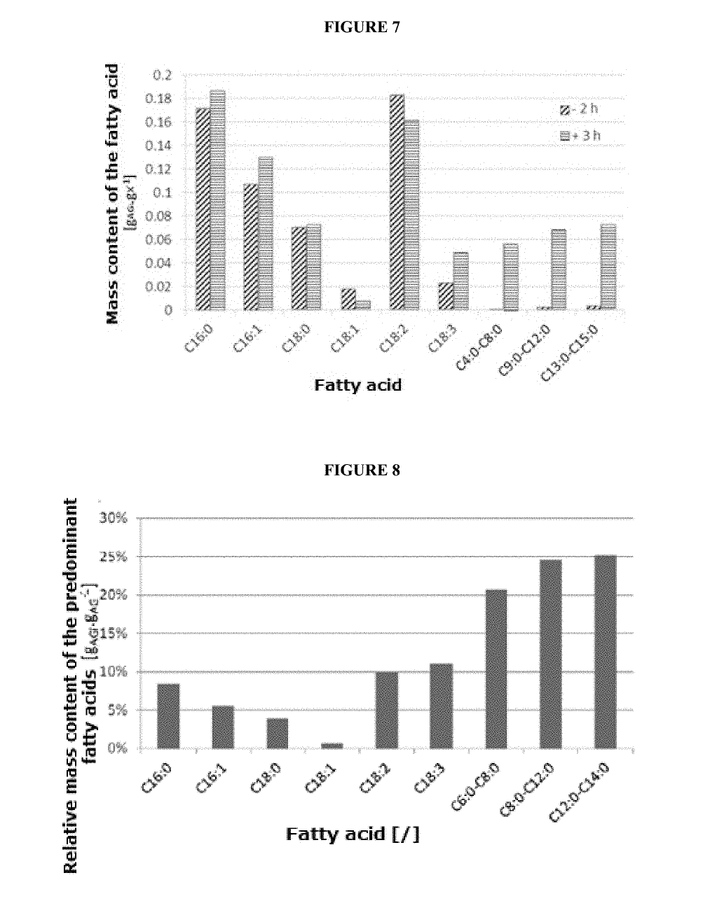

FIG. 7: Development of the mass content [g_{AGi}·gₓ⁻¹] of the different fatty acids predominantly present in Y. lipolytica during the phase of lipid accumulation before (−2 h) and after (3 h) a pulse of 7 mg_{cerulenin}·gₓ⁻¹ during a fed-batch culture. (Culture B)

FIG. 8: Profile of the fatty acids accumulated by Y. Lipolytica 2 h after a pulse of cerulenin of 7 mg_{cerulenin}·gₓ⁻¹ during a fed-batch culture with nitrogen limitation (Culture B).

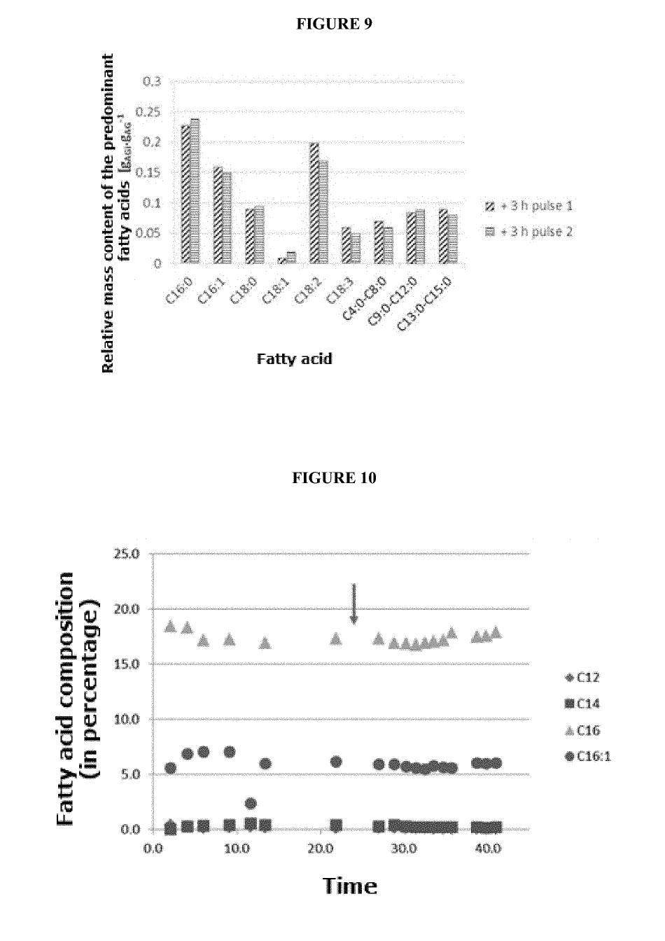

FIG. 9: Comparison of the fatty acid profiles during the phase of lipid accumulation 3 h after pulses 1 and 2 during a fed-batch culture of Y. lipolytica. (Culture B).

FIG. 10: Development of the fatty acid profile before and after a pulse of ethanol during a fed-batch culture of Y. lipolytica JMY3501 (Culture C). The arrow indicates the point in time at which the pulse of ethanol was provided.

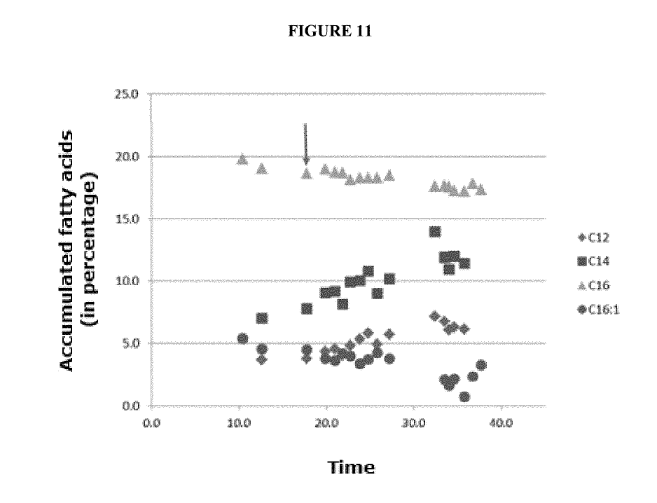

FIG. 11: Development of the fatty acid profile before and after a pulse of 0.25 mg_{cerulenin}·gₓ⁻¹ during a fed-batch culture of Y. lipolytica JMY3501 (Culture D). The arrow indicates the point in time at which the pulse of cerulenin was provided.

### A—PRACTICAL EXAMPLES OF THE INVENTION WITH THE STRAIN OF YARROWIA LIPOLYTICA W29

### 1—Materials and Methods

1.1—Strain Yarrowia lipolytica W29 and Culture Media

The strain Yarrowia lipolytica W29 is a wild-type strain. Stocks of the strain were produced from axenic pre-cultures produced in baffled Erlenmeyer flasks placed on a rotary stirring table, with a rich medium having an initial concentration of glucose of 10 g/L. In the middle of the exponential phase, samples of 1 mL were taken and mixed with sterile glycerol (30% volume/volume). These stocks were then stored in sterile vials at −80° C. These frozen concentrated cultures were used to seed the various pre-cultures for the purposes of fed-batch culture. The pre-cultures of yeast were performed in two 100 mL Erlenmeyer flasks containing 8 mL of rich medium LB at 30° C. for 16 h on a rotary stirring table (100 rpm). The cultures were transferred to two 250 mL Erlenmeyer flasks containing 72 mL of mineral medium (pH 5.6) having an initial concentration of glucose of 10 g/L. After 12 h at 30° C., the cultures of 80 mL volume were used to seed two 5 L Erlenmeyer flasks containing 710 mL of mineral medium with vitamins. These flasks were incubated at 30° C. for 12 h, with an initial concentration of glucose of 10 g/L. The content of one of the flasks from the last culture was used to seed 8 L of mineral medium in a 20 L bioreactor. The series of pre-cultures performed in parallel was used to check the reproducibility of the pre-cultures.

The composition of the medium of type LB was as follows: casein peptone 10 g/L; NaCl 9 g/L; autolysed yeast extract 5 g/L with glucose at a concentration of 10 g/L.

The composition of the mineral medium was as follows: K₂HPO₄: 3 g/L; (NH₄)₂SO₄: 3 g/L; NaH₂PO₄,H₂O: 3 g/L; MgSO₄,7H₂O: 1 g/L; ZnSO₄,7H₂O: 0.04 g/L; FeSO₄,7H₂O: 0.0163 g/L; MnSO4,H₂O: 0.0038 g/L; CoCl₂,6H₂O: 0.0005 g/L; CuSO₄,5H₂O: 0.0009 g/L; Na₂MoSO₄,2H₂O: 0.00006 g/L; CaCl₂,2H₂O: 0.23 g/L; H₃BO₃: 0.03 g/L; and 10 mL of a solution of vitamins. The solution of vitamins had been prepared at the following concentration by a factor of 1000: d-biotin: 0.05 g/L, thiamine chlorohydrate: 1 g/L, panthotenic acid: 1 g/L, pyridoxol chlorohydrate: 1 g/L; nicotinic acid: 1 g/L, p-aminobenzoic acid: 0.2 g/L, myo-inositol: 25 g/L. Before sterilisation, the pH of this medium was adjusted to 4.5 with a solution of H₃PO₄ and to a working pH (5.5) with an ammonia solution.

1.2—Cultures

Fed-batch cultures (8 L) were produced in a 20 L bioreactor (total volume) using the Braun Biostat E culture system (Braun, Melsungen, Germany) without oxygen limitation.

The temperature was controlled to 28° C. and the pH to 5.5 by addition of a 10 mol/L solution of NH₃ (growth phase) or of a solution of KOH (lipid accumulation phase). A software developed in the laboratory of the inventors made it possible to acquire and control the operating parameter values, such as the stirring speed, pH, temperature, partial pressure of dissolved oxygen (DO), and volumes and flow rates of the feed of the bases and of the anti-foaming agent.

The pressure in the bioreactor was regulated to 0.3 bar (relative pressure).

The maximum amount of anti-foaming agent (Struktol) added was equal to 0.5 mL per culture.

The bioreactor was equipped with three sterile feed systems (carbon source advantageously from glucose alone, salt, ammonia or potassium hydroxide) using peristaltic pumps (Masterflex and Gilson). The concentration of the feed of carbon source, advantageously from glucose alone, was equal to 730 g/L. The masses of the carbon source solution and of the ammonia (or potassium hydroxide) solution introduced into the bioreactor were measured continuously by monitoring the masses of the flasks containing the solution stocks (Sartorius scales). The concentrations of carbon source and of nitrogen in the fermenter were estimated according to the carbon balance and redox balance equations. The rate of evaporation was estimated on the basis of the culture temperature, the efficacy of the condenser of the fermenter, and the aeration flow rate. The culture volume was calculated according to a material balance realised on the basis of the inputs of substrate, salt, ammonia, base, vitamins and anti-foaming agent and the outputs by evaporation and sampling with and without biomass.

1.3—Chemical Agents

The chemical products (glycerol, salts, oligoelements, orthophosphoric acid and NH₃) were provided by Prolabo (France), and the vitamins were provided by Sigma (E.U.A.). All of these products were of the highest analytical quality available. The cerelose for the fed-batch cultures was provided by Roquette (France).

1.4—Strategy for Feeding Glucose

During the growth phase, an exponential profile of the flow rate of the pump feeding the carbon source made it possible to maintain a constant growth rate.

During the phase of accumulation, a constant growth rate was maintained in the most stable manner possible by an exponential flow rate of the carbon source.

1.5—Strategy for Feeding Concentrated Salts

The bioreactor was fed by a flow rate of a solution of concentrated salts corresponding to 1/10 of the flow rate feeding the substrate. The composition of the solution of concentrated salts was as follows: KCl: 20 g/L, CuSO₄,5H₂O: 0.6 g/L, NaCl: 20 g/L, Na₂MoO₄,2H₂O: 0.094 g/L, MgSO₄,7H₂O: 27 g/L, CaCl₂,2H₂O: 6.4 g/L, ZnSO₄,7H₂O: 7.7 g/L, FeSO₄,7H₂O: 3.97 g/L, MnSO₄,H₂O: 0.47 g/L, H₃BO₃: 0.3 g/L, CoCl₂,6H₂O: 0.3 g/L, H₃PO₄: 46.7 g/L.

1.6—Strategy for Feeding Vitamins

All of the cultures were performed with a sequenced feed of vitamins as a function of the growth rate: quantities of 0.1% (vol/vol) of solution of vitamins were added during production of 10 g/L of biomass.

1.7—Strategy for Feeding Ammonium

During the growth phase, the nitrogen was added with the aid of the base pump in order to regulate the pH to a constant value equal to 5.5. During the lipid production phase, the addition of nitrogen was controlled by a peristaltic pump with an exponential flow rate, varying from 0.00014 L·h⁻¹ to 0.004 L·h⁻¹, of solution of NH₃ (5 mol/L) in order to maintain a constant specific growth rate; the pH was regulated by addition of a solution of KOH (10 mol/L).

### 2. Analytical Methods

2.1—Quantification and Qualification of the Biomass

The concentration of yeast was determined by spectrophotometric measurements at 600 nm in a HITACHI U-1100 spectrophotometer in a quartz cell having an optical path of 0.2 cm. Dilutions of the sample were performed such that the optical density was within the range of 0.1 to 0.6 AU. For each sample, the average of three measurements was calculated. In order to determine the dry mass of the cells, culture samples (5 to 10 ml) were collected by filtration over a 0.45 mm membrane (Sartorius) and were dried at 200 mm Hg and 60° C. for 48 h until a constant mass was obtained.

An on-line estimation of the concentration of active cells was performed using a capacitance probe (Fogale). This technology is based on the correlation between the volume of the viable catalytic biomass and the variation of the dielectric permittivity of the medium in which the cells are dispersed.

All the cell concentrations were expressed in gₘₛ/L, that is to say the dry mass of yeast per unit of volume of culture. The amount of ash was determined after two processes of total combustion of the dry mass filters with biomass in the presence of 200 mL of 20 g/L solution of NH₄NO₃ in a muffle furnace at 550° C. for 12 h each time. The formula of the biomass was determined at ENSIACET (Toulouse, France) by elementary analysis of C, H, O and N and the ashes. Due to a significant accumulation of lipids, the formulas of the biomass varied during the course of the culture from CH_{1.86}O_{0.52}N_{0.13} (growth phase) to CH_{2.00}O_{0.59}N_{0.07} (accumulation phase).

2.2—Sampling

Every 20 minutes, a sample of supernatant was collected by a tangential filtration system connected to an automated fraction collector. A sample of culture medium was collected every hour directly by means of a septum. All of the samples were stored at −20° C.

2.3—Analysis of the Outlet Gases of the Reactor

The outlet gases of the fermenter were analysed every 20 seconds by mass spectroscopy at the outlet of the gas condenser of the fermenter. The mass spectrometer (PRIMA 600s; VG Gas, Manchester, United Kingdom) was used due to its accuracy in measuring the compositions of CO₂, O₂, N₂ and Ar.

The rate of O₂ consumption and the rate of CO₂ production were calculated according to the material balances, combining the volume of the gases in the reactor, the flow rate of inflowing air (measured by a mass flowmeter), the temperature, humidity, and the pressure and composition of the inlet and outlet gases.

2.4—Extraction and Quantification of the Lipids

The total cellular lipids were extracted in accordance with the technique of Cescut J. et al. (PloS one; 6 (11): e27966, 2011), which is an automisation of the working method of Bligh and Dyer, as follows: the gradient extraction of solvent was performed in a pressurised liquid extractor (SPE). 500 mg of lyophilisates were placed in the extraction cells. Three different solvent mixtures were injected under pressure and heat into the cell (100° C., 100 bars). The successive solvent mixtures were: methanol/chloroform (2:1, vol/vol), (1:1, vol/vol) and lastly (1:2, vol/vol).

The three organic phases were mixed and washed twice with a 25% (vol/vol) solution of a 0.88% solution of KCl (mass/volume) for 15 minutes under gentle stirring. The organic phase was recovered by liquid/liquid separation after centrifugation (5000×g, 10 min).

Lastly, the lipids were collected after the evaporation of the solvents in a centrifugal evaporator (45° C.; 500 g) from the Genevac brand. The total content of lipids was quantified by a gravimetric method. The extract of lipids was held in a chloroform/methanol mixture at −20° C.

2.5—Evaluation of the Fatty Acid Profiles

The free or bonded fatty acids were methylated in fatty acid methyl ester (FAME) using trimethylsufonium hydroxide (TMSH, 0.2 M in methanol, Macherey-Nagel, Germany). The analysis was performed using a Hewlett-Packard 5890 gas phase chromatography apparatus equipped with a WCOT fused silica column measuring 50 m×250 mm×25 mm in size (VARIAN, E.U.A.) and equipped with an FID, under the following conditions: mobile phase: N₂, flow rate 50 mL·min⁻¹, temperature of the furnace: 50-75° C. at 9° C.·min⁻¹, then 75-140° C. at 13° C.·min⁻¹, then 140-180° C.·min⁻¹ at 1.5° C.·min⁻¹, then 180-240° C. at 4.5° C.·min⁻¹, injector temperature 140° C., detector temperature 250° C.

### 3. Results

According to preliminary studies, in which the mass of cerulenin (antibiotic) per mass unit of biomass (x) varied between 1 mg_{cerulenin}·gₓ⁻¹ and 25 mg_{cerulenin}·gₓ⁻¹, it was found that a dose of 15 mg_{cerulenin}·gₓ⁻¹ completely inhibits growth. A dose of 7 μg_{cerulenin}·mgₓ⁻¹ was thus retained for partial inhibition of the growth and the quantification of the modulation of the rate of elongation of the fatty acids of Y. lipolytica. Two cultures were performed.

Culture A, referred to as the control culture, made it possible to identify the influence of the DMSO, solvent of the antibiotic, on the physiology of the yeast. DMSO is a solvent which is indispensable for dissolving the antibiotic that was added during a pulse in the culture B.

All the operating conditions were identical during these two cultures.

### Culture A

The results of culture A are shown by FIG. 2 and FIG. 3; they show the development of the growth and of the fatty acid profiles as a function of the culture time. It would appear that the growth dynamic is not influenced by the injection of DMSO. The stability of the fatty acid profile during the period between +15 min and +3 h relative to the DMSO pulse completes the analysis, revealing that DMSO does not affect the metabolism of lipid accumulation.

Conclusion: In control culture A, the DMSO pulse influences neither the rate of growth of the yeast nor the fatty acid profile.

### Culture B

A pulse of cerulenin was introduced into the culture B at 28.2 h (FIG. 4), i.e. 11 h after the start of the nitrogen limitation phase, this pulse triggering the induction of lipid biosynthesis with a growth rate maintained at 0.045 h⁻¹ (maximum variation 5%), when a cell concentration of 6.9 gₓ·L⁻¹ was reached.

With regard to the development of the cell concentration over time, the growth dynamic was not influenced by the cerulenin pulse during the 10 h of culture following the injection. As shown in FIG. 5, the variation of the growth rate during the 10 h following the first injection of cerulenin was less than 5%. It is shown that the supply of a cerulenin dose of 7 μg_{cerulenin}·mgₓ⁻¹ during a culture of Y. lipolytica under nitrogen limitation conditions had no effect on the growth dynamic of the yeast.

Throughout the nitrogen limitation phase, an accumulation of total fatty acids was quantified on the basis of the imposed flow rate of the substrate in accordance with previous works (Cescut et al., PloS one; 6 (11): e27966, 2011) with an absence of citric acid secretion: 20% total fatty acids were accumulated during the entire nitrogen limitation phase (50 h), 3% of which were accumulated during the 10 h following the cerulenin pulse. With regard to the kinematic behaviour, a reduction of the specific speed of production of fatty acid from 0.004 g_{AG}·gₓ·h⁻¹ to 0.0017 g_{AG}·gₓ·h⁻¹ was observed following the addition of cerulenin.

With regard to the lipid profile, significant developments of the fatty acid composition of the lipids accumulated before and after the cerulenin pulse were observed. Looking at 0 h, the time of injection (culture time 28.2 h), it would appear that the fatty acid profile before the addition of cerulenin is composed primarily of C16:1 (17%), C18:2 (31%) and C16:0 (29%) with short-chain or medium-chain fatty acid levels (less than 15 carbon atoms) being less than 1%. The degree of unsaturation, defined by the ratio between the number of moles of unsaturation and the number of fatty acid moles, is 0.95⁺/₋2% and the average length of the carbon chain, defined by the average carbon number of all the fatty acids, is 16.98⁺/₋2%.

After the cerulenin pulse, from 15 min, the appearance of short-chain fatty acids was observed. This accumulation of fatty acid reached 24.5% after 3 h of culture. This was an unexpected result.

Between the injection and 3 h after the injection of cerulenin, Y. lipolytica synthesised and accumulated neo-synthesised fatty acids with an average degree of unsaturation of 0.6 and an average number of carbon atoms of 12.74 carbon atoms (Table 1).

The mass contents of fatty acids with a carbon chain length of C4:0-C8:0, C9:0-C12:0 and C13:0-C15:0 increased on the basis of the cerulenin pulse: the variation of mass in relation to the lipid composition prior to the pulse reached, respectively, 0.05 g_{AG}·gₓ⁻¹, 0.07 g_{AG}·gₓ⁻¹ and 0.07 g_{AG}·gₓ⁻¹ in 3 h for the three aforementioned groups (FIG. 6). By contrast, the mass content of palmitic acid (C16:0) increased from 0.014 g_{AG}·gₓ⁻¹ and that of palmitoleic acid (C16:1) from 0.023 g_{AG}·gₓ⁻¹ in relation to the lipid composition before the pulse (FIG. 7). The specific rate of synthesis of the short-chain or medium-chain fatty acid is multiplied by a factor of 14 when the dynamics before and 3 h after the pulse are compared.

By defining F_{n,p} as the mass fraction of a group of fatty acids of carbon chain length Cₙ to Cₚ relative to the total mass of accumulated fatty acid, it would appear that F_{4,8} is multiplied by 70 at 3 h, F_{9,12} by 28 and F_{13,15} by 15. For the fatty acids with a chain length greater than 15, a significant reduction of the mass fraction of fatty acids C16:0 and C18:2 was observed, whereas the mass fraction of the fatty acid C18:3 rose to 6% (FIG. 8). This is translated with regard to the degree of unsaturation into a reduction from 0.95 to 0.75 in 3 h and a reduction of the length of the carbon chain from 16.98 to 15.3.

TABLE 1
Degree of unsaturation and length of the carbon chains of free
or esterified fatty acids present in Y. lipolytica during the phase of
lipid accumulation before, during, and after a pulse of 7 mg_{cerulenin} ·
gₓ⁻¹ during the course of a fed-batch culture of Y. lipolytica.
(Culture B)

| | −2 h | 0 h | +15 min. | +1 h | +3 h |
|---|---|---|---|---|---|
| Degree of unsaturation | 0.97 | 0.95 | 0.92 | 0.73 | 0.75 |
| Length of the carbon chain | 16.97 | 16.98 | 16.83 | 16.3 | 15.3 |

The effect of the partial inhibition of the elongation kinetics of the fatty acids by the cerulenin pulse disappeared after 9 h of culture: the fatty acid profile became identical to the profile of accumulated fatty acids before the pulse.

A complementary experiment in which a second pulse was introduced made it possible to reproduce the same biological phenomena as after the first pulse. The fatty acid profiles 3 h after pulses 1 and 2 are illustrated in FIG. 9. They are both similar for all fatty acids.

### Conclusions

A dose of cerulenin of 7 mg_{cerulenin}·gₓ⁻¹ makes it possible, during the lipid synthesis phase in Y. lipolytica on glucose in fed-batch mode:
□ to maintain the growth dynamic and the synthesis of lipids,
□ to produce an accumulation of short-chain fatty acids (C4-C15) by partial inhibition of the elongation kinetics of the fatty acids.

A strategy of sequenced additions of cerulenin doses has proven to be indispensable for maintaining the modulation of the profile of fatty acids synthesised by Y. lipolytica by encouraging the accumulation of short-chain or medium-chain fatty acids.

### B—PRACTICAL EXAMPLES IN ACCORDANCE WITH THE INVENTION WITH THE STRAIN OF YARROWIA LIPOLYTICA JMY3501

### 1—Materials and Methods

### Strain and Culture

The strain of Yarrowia lipolytica JMY3501 is a strain genetically modified so as to optimise the accumulation of lipids, the culture conditions and the conditions for obtaining said strain being described in (Lazar Z et al., Metabolic Engineering 26 (2014) 89-99).

The strain of Yarrowia lipolytica JMY3501 can be prepared for example by deriving the strain JMY1233 (Beopoulos et al., Applied and Environmental Microbiology 74 (2008) 7779-7789) as follows:
i. TGL4 is deactivated by introducing the tgl4::URA3ex disruption cassette from the strain JMP1364 (Dulermo et al., Biochimica et Biophysica Acta 1831 (2013) 1486-1495), which produces the strain JMY2179.
ii. An auxotrophic marker, URA3ex, is then removed from the strain JMY2179 using the strain JMP547 (Fickers et al., Journal of Microbiological Methods 55 (2003) 727-737), which produces the strain JMY3122.
iii. The strain JMY3501 is then obtained by introducing, successively to the strain JMY3122, pTEF-DGA2-LEU2ex from the strain JMP1822, and pTEF-GPD1-URA3ex from the strain JMP1128 (Dulermoz and Nicaud, Metabolic Engineering 13 (2011) 482-491). The strain JMP1822 is obtained by replacing the marker URA3ex of the strain JMP1132 (Beopoulos et al. (Beopoulos et al., Applied and Environmental Microbiology 74 (2008) 7779-7789) with LEU2ex.

### Culture C

Culture C was performed in fed-batch mode with the Yarrowia lipolytica yeast strain JMY3501, in a 3 L bioreactor with a usable volume of 1.5 L using the Biostat B. Braum Biotech International culture system (Sartorius AG, Germany) with the acquisition software MFCS/win 2.0. The temperature was regulated to 28° C. and the pH was regulated by addition of a 2.5 mol/L solution of NH₄OH for the growth phase and by addition of a 2.5 mol/L solution of KOH for the nitrogen limitation phase. With the aim of avoiding an oxygen limitation, the amount of inflowing air and the stirring speed were controlled so as to keep the dissolved oxygen above 20% saturation. The compositions of the inflow and outflow air were analysed with the aid of a mass spectrometer (Amatek Process Instruments).

### Culture D

The objective of culture D was to study the impact of cerulenin pulses, in a ratio less than 1 mg/g of dry mass of biomass, on the metabolism of the Yarrowia lipolytica yeast strain JMY3501 in terms of lipid accumulation, fatty acid composition, and citric acid production.

Culture D was performed under the same culture conditions as culture C.

The solution of cerulenin was prepared in ethanol and a pulse of 0.25 mg_{cerulenin}·gₓ⁻¹ was introduced 6 h after the triggering of the nitrogen limitation phase.

### 2—Result

### Culture C

The results of culture C are shown in FIG. 10; they show the development of the fatty acid profile as a function of the culture time. It would appear that the fatty acid profile is stable before and after the ethanol pulse, indicating that ethanol does not affect the metabolism of lipids.

### Culture D

A significant development of the fatty acid composition of the lipids accumulated before and after the cerulenin pulse (FIG. 11) can be seen. Following the cerulenin pulse, there appears to be an increase in short-chain fatty acids, primarily of C14 and C12. Before the cerulenin pulse, the C14 content in the fatty acid composition was 7%, and that of C12 was 4%, whereas after the cerulenin pulse the C14 content was 14% and that of C12 was 7%.

It would appear that a cerulenin dose of 0.25 mg_{cerulenin}·gₓ⁻¹ makes it possible to increase the accumulation of short-chain fatty acids during the lipid accumulation phase in Yarrowia lipolytica JMY3501.

1. Method for synthesis of short-chain or medium-chain fatty acids by culturing a eukaryotic microorganism from the kingdom of fungi, that is naturally oleaginous or originates from the yeast strain JMY3501, wherein the culture is performed in the presence of a fatty acid synthase inhibitor in the culture medium.

2. Method for synthesis of short-chain or medium-chain fatty acids by culturing a eukaryotic microorganism from the kingdom of fungi, that is naturally oleaginous, wherein the culture is performed in the presence of a fatty acid synthase inhibitor in the culture medium.

3. Method according to claim 1, wherein the short or medium chain of the fatty acids has between 4 and 15 carbon atoms.

4. Method according to claim 1, wherein the fatty acid synthase inhibitor is selected from cerulenin and analogues thereof, triclosan (5-chloro-2-(2,4-dichlorophenoxy) phenol), TOFA (5-(tetradecyloxy)-2-20 furancarboxylic acid), bischloroanthrabenzoxocinone, thiolactomycin, platensimycin and also the analogues of these molecules selected from C75 (4-methylene-2-octyl-5-oxo-tetrahydrofuran-3-carboxylic acid), C93 (or FAS93), FAS31, orlistat (N-formyl-L-leucine (1S)-1-[[(2S,3S)-3-hexyl-4-oxo-2-oxetanyl]methyl]dodecyl ester), GSK837149A (dibenzenesulfonamide urea), isoniazid, platencin, pyrazinamide, ethionamide, diazoborine, hexachlorophene, diclofenac, epigallocatechin-3-gallate (EGCG), luteolin, taxifolin, kaempferol, quercetin, apigenin, anthecotulide, anthecularin, 4-hydroxyanthecotulide, 4-acetoxyanthecotulide, and C247.

5. Method according to claim 1, wherein the fatty acid synthase inhibitor is cerulenin.

6. Method according to claim 1, wherein the microorganism is of the Yarrowia, Saccharomyces, Rhodotorula, or Rhodosporidiu genus.

7. Method according to claim 6, wherein the microorganism is the yeast Yarrowia lipolytica or Rhodotorula glutinis.

8. Method according to claim 6, wherein the microorganism is the yeast Yarrowia lipolytica.

9. Method according to claim 4, wherein the cerulenin is introduced into the culture medium either by continuous addition, or by pulsed addition, one-time addition, or multiple and successive additions.

10. Method according to claim 9, wherein the concentration of cerulenin varies from 0.01 to 25 mg/g of dry yeast.

11. Method according to claim 9, wherein the concentration of cerulenin varies from 1 to 25 mg/g of dry yeast.

12. Method according to claim 9, wherein the concentration of cerulenin varies from 0.01 to 1 mg/g of dry yeast.

13. Method according to claim 1, wherein the ratio between the rate of carbon consumption and the rate of nitrogen consumption (rC/rN) has a value between 5 and 100 moles of carbon consumed per mole of nitrogen consumed.

14. Method according to claim 1, wherein the ratio between the rate of carbon consumption and the rate of nitrogen consumption (rC/rN) has a value between 16 and 100 moles of carbon consumed per mole of nitrogen consumed.

15. Method according to claim 1, wherein the ratio between the rate of carbon consumption and the rate of nitrogen consumption (rC/rN) has a value between 12 and 50 moles of carbon consumed per mole of nitrogen consumed.

16. Method according to claim 1, wherein the content of phosphorus in the culture medium could be adjusted so as to keep the level of intracellular phosphorus of the yeast at a value varying from 4 to 27 mg/g of biomass.

17. Method according to claim 1, wherein the short-chain or medium-chain fatty acids are obtained in the form of a mixture of free fatty acids and triglycerides.

18. Method according to claim 9, wherein the concentration of cerulenin varies from 0.01 to 14 mg/g of dry yeast.

19. Method according to claim 9, wherein the concentration of cerulenin varies from 0.05 to 14 mg/g of dry yeast.

20. Method according to claim 9, wherein the concentration of cerulenin varies from 1 to 14 mg/g of dry yeast.
